(19) United States
(12) Patent Application Publication
(10) Pub. No.: US 2026/0215825 A1
(43) Pub. Date: Jul. 30, 2026

(54) INDEPENDENT SCREW TARGETING AROUND INTRAMEDULLARY NAIL

(71) Applicant: DEPUY SYNTHES PRODUCTS, INC., Raynham, MA (US)

(72) Inventor: Stanley KMIEC, Morgantown, PA (US)

(21) Appl. No.: 19/422,986

(22) Filed: Dec. 17, 2025

Related U.S. Application Data
(60) Provisional application No. 63/750,128, filed on Jan. 27, 2025.

(51) Int. Cl. A61B 17/72; A61B 17/88; A61B 17/90; A61B 17/56

(52) U.S. Cl. CPC A61B 17/7291; A61B 17/8872; A61B 17/8897; A61B 17/90; A61B 2017/564

(57) ABSTRACT

A system includes a nail, an aiming jig having connecting hole and openings, and an inserting handle releasably coupled to the nail and the jig. The nail is inserted through a medullary canal of a tibia such that a distal portion of the nail is received within a calcaneus and a talus of a foot while a proximal portion of the nail is received within the tibia. The hole is sized, shaped and slidably receives a distal portion of the handle so that the jig is in a desired orientation relative to the handle and the nail. The openings extend through the jig along central axes defining a trajectory along which independent fixation elements are inserted through to provide supplemental fixation of subtalar joint or tibiotalar joint without colliding with the nail and locking elements received within holes of the nail when the jig is in an operative configuration.

### PRIORITY CLAIM

The present application claims priority to U.S. Provisional Patent Application Serial No. 63/750,128 filed January 27, 2025; the disclosure of which is incorporated herewith by reference.

### BACKGROUND

Tibi talocalcaneal (TTC) arthrodesis is a procedure involving fusion of the tibiotalar joint and the subtalar joint (talocalcaneal joint) to alleviate pain resulting from, for example, arthritic hindfoot or ankle joint, and/or to correct joint deformities. During a TTC fusion procedure, a nail is inserted into a medullary canal of a tibia via a plantar aspect of the foot so that the nail extends through the calcaneus, talus, and tibial bones. The nail may then be fixed relative to the bones via a plurality of locking screws. In some cases, it may be desired to supplement the nail fixation using additional independent screws. Currently, insertion of the independent screws is achieved via a freehand technique that requires the surgeon to select a trajectory of the independent screws which would avoid colliding with the intramedullary nail and the locking screws. This freehand technique may be challenging and time consuming.

### SUMMARY

The present disclosure relates to an intramedullary nail system for providing a tibiotalocalcaneal (TTC) fusion. The system includes an intramedullary nail configured to be inserted through a medullary canal of a tibia such that a distal portion of the intramedullary nail is received within a calcaneus and a talus of a foot while a proximal portion of the intramedullary nail is received within the tibia, the intramedullary nail extending longitudinally from a distal end to a proximal end and including a plurality of locking holes extending transversely therethrough; an insertion handle configured to facilitate insertion of the intramedullary nail into the medullary canal of the tibia, the insertion handle extending from a distal end to a proximal end releasably couplable to the distal end of the intramedullary nail; and an aiming jig configured to be releasably coupled to the insertion handle, the aiming jig including a connecting hole and a plurality of openings extending therethrough, the connecting hole being sized, shaped and configured to slidably receive a distal portion of the insertion handle therethrough so that the aiming jig is in a desired orientation relative to the insertion handle and the intramedullary nail, the plurality of openings extending through the aiming jig along central axes defining a trajectory along which independent fixation elements are configured to be inserted through to provide supplemental fixation of one of a subtalar joint and a tibiotalar joint of the foot without colliding with the intramedullary nail and locking elements received within the locking holes thereof, when the aiming jig is in an operative configuration.

In an embodiment, the aiming jig includes a first portion extending along a first plane and a second portion extending along a second plane that is angled with respect to the first plane.

In an embodiment, the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

In an embodiment, the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.

In an embodiment, the aiming jig is symmetrical about a midplane, which extends perpendicular to the first plane and the second plane and through a midline of the connecting hole.

In an embodiment, the aiming jig includes a collar extending from the surface of the first portion, the collar having a channel that is coaxially aligned with the connecting hole.

In an embodiment, the connecting hole is keyed to the distal portion of the insertion handle such that the aiming jig is slidable therealong in the desired orientation.

In an embodiment, the aiming jig includes a locking hole extending through a portion thereof in communication with the connecting hole, the locking hole configured to receive a lock screw therein to lock the aiming jig relative to the insertion handle, in the operative configuration.

In an embodiment, the system further includes a guide sleeves sized, shaped and configured to be inserted through any one of the plurality of openings, the guide sleeve extending longitudinally from a proximal end to a distal end and including a channel extending therethrough, the channel of the guide sleeve configured to slidably receive a guidewire therethrough.

In addition, the present disclosure relates to an exemplary method for treating bones, comprising: inserting an intramedullary nail through a medullary canal of a tibia, using an insertion handle, via a plantar aspect of a foot so that a distal portion of the intramedullary nail extends through a talus and calcaneus of the foot and a proximal portion extends through the tibia; inserting locking elements through locking holes extending through a distal portion and a proximal portion of the intramedullary nail to fix the intramedullary nail relative to the calcaneus, talus, and tibial bones; releasably coupling an aiming jig to a distal portion of the insertion handle so that the aiming jig is in an operative configuration relative to the foot, wherein coupling the aiming jig relative to the insertion handle includes sliding the aiming jig proximally along the distal portion of the insertion handle such that the insertion handle is received within a connecting hole of the aiming jig; and inserting an independent fixation element through a selected one of a plurality of openings extending through the aiming jig, each opening of the plurality of openings extending through the aiming jig along a central axis having a trajectory targeting one of a subtalar joint and a tibiotalar joint of the foot without interfering with the intramedullary nail and the locking elements extending therethrough.

In an embodiment, the method further includes inserting a guide sleeve through the selected one of the plurality of openings until a proximal end of the guide sleeve abuts the calcaneus; and inserting a guidewire through the guide sleeve and into the foot to a desired distance so that the guidewire extends along the trajectory of the selected one of the plurality of openings, wherein inserting the independent fixation element includes removing the guide sleeve and sliding the independent fixation element along the guidewire and into the foot, across one of the subtalar and tibiotalar joints.

In an embodiment, the method further includes locking the aiming jig in the operative configuration relative to the insertion handle by inserting a lock screw through a locking hole of the aiming jig, the locking hole of the aiming jig extending through a portion of the aiming jig such that the locking hoe is open to and in communication with the connecting holes.

In an embodiment, the aiming jig is slid along the distal portion of the insertion handle by gripping a collar of the aiming jig, the collar extending from a surface thereof and having a channel that is coaxially aligned with the connecting hole.

In an embodiment, the aiming jig includes a first portion extending along a first plane and a second portion extending along a second plane that is angled with respect to the first plane.

In an embodiment, the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

In an embodiment, the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.

In an embodiment, the aiming jig is symmetrical about a midplane, which extends perpendicular to the first plane and the second plane and through a midline of the connecting hole.

In addition, the present disclosure relates to a device for providing supplemental fixation during a tibiotalocalcaneal (TTC) fusion. The device includes an aiming jig configured to be releasably coupled to an insertion handle for inserting an intramedullary nail through a medullary canal of a tibia, the aiming jig including a connecting hole and a plurality of openings extending therethrough, the connecting hole being sized, shaped and configured to slidably receive a distal portion of the insertion handle therethrough so that the aiming jig is in a desired orientation relative to the insertion handle and the intramedullary nail, the plurality of openings extending through the aiming jig along central axes defining a trajectory along which independent fixation elements are configured to be inserted through to provide supplemental fixation of one of a subtalar joint and a tibiotalar joint of a foot without colliding with the intramedullary nail and any locking elements received within locking holes thereof, when the aiming jig is in an operative configuration.

In an embodiment, the aiming jig includes a first portion extending along a first plane and a second portion extending along a second plane that is angled with respect to the first plane.

In an embodiment, the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

In an embodiment, the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.

### BRIEF DESCRIPTION

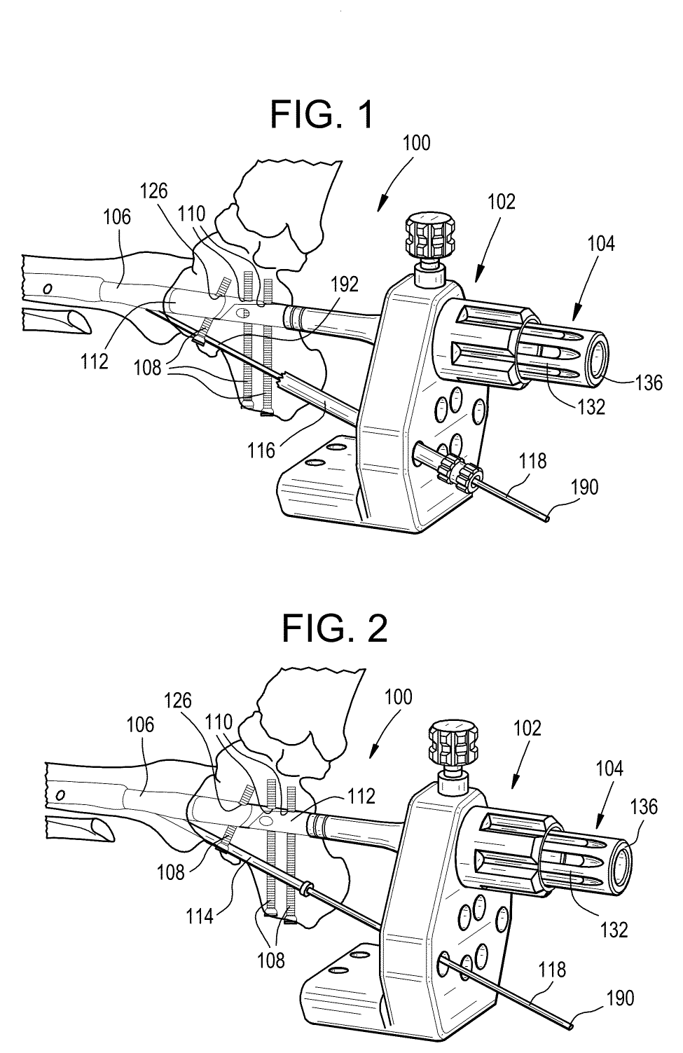

FIG. 1 shows a perspective view of a system according to an exemplary embodiment of the present disclosure;

FIG. 2 shows another perspective view of the exemplary system according to FIG. 1;

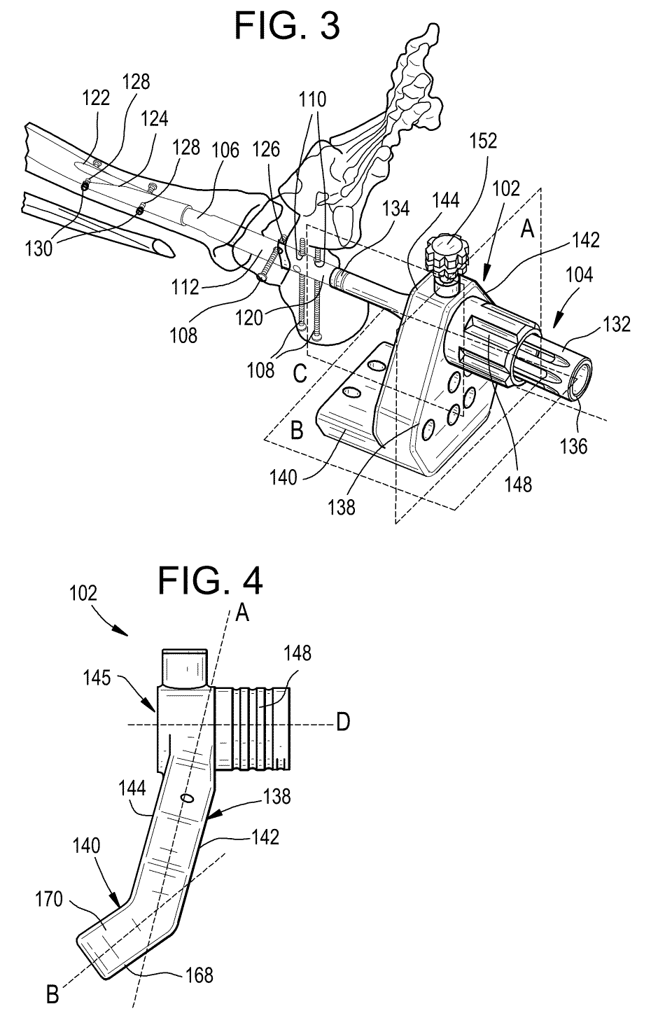

FIG. 3 shows yet another perspective view of the exemplary system according to FIG. 1;

FIG. 4 shows a plan view of an aiming jig according to the exemplary system of FIG. 1, in a proximal direction, perpendicular to a plane A;

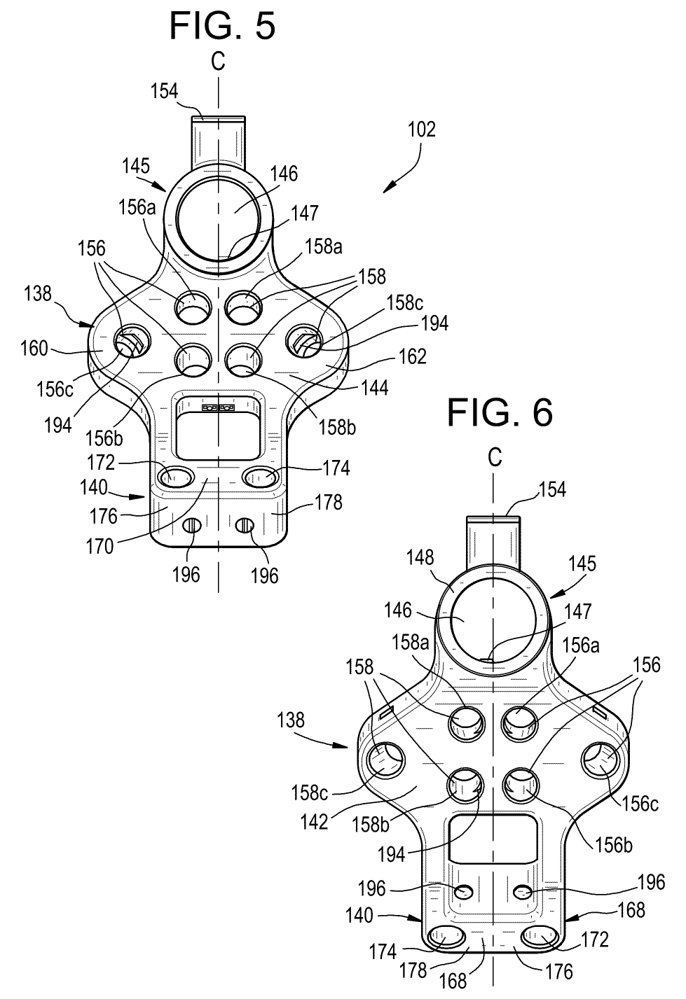

FIG. 5 shows another plan view of the aiming jig according to the exemplary system of FIG. 1, in a posterior direction perpendicular to plane B;

FIG. 6 shows another plan view of the aiming jig according to the exemplary system of FIG. 1, in an anterior direction perpendicular to plane B;

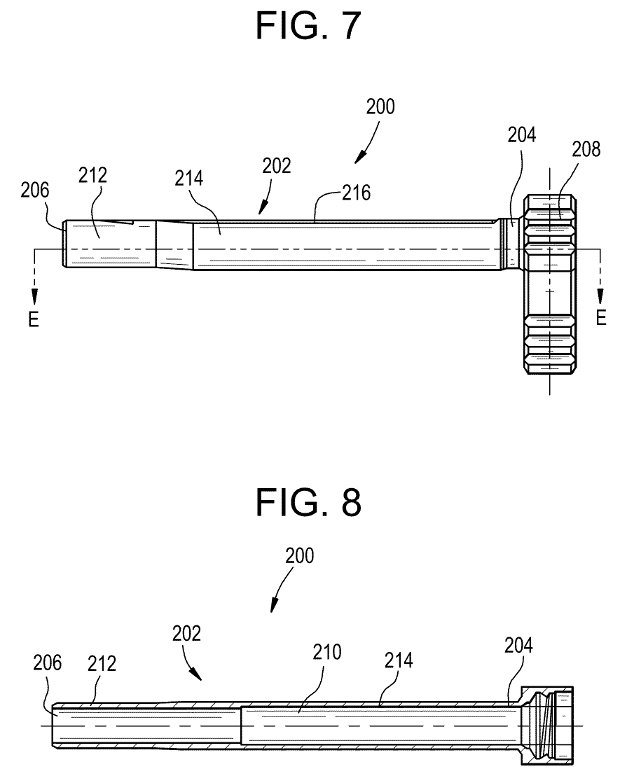

FIG. 7 shows a longitudinal side view of a protection sleeve according to an exemplary system of the present disclosure;

FIG. 8 shows a longitudinal cross-sectional view of the protection sleeve of FIG. 7, along a line E-E;

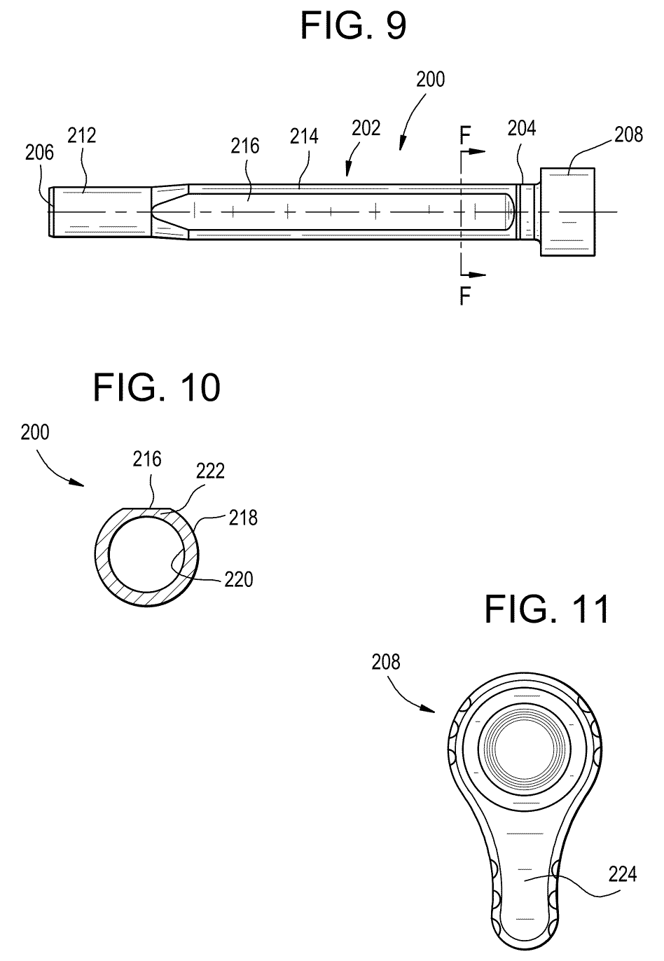

FIG. 9 shows another longitudinal side view of the protection sleeve of FIG. 7, rotated 90 degrees about a longitudinal axis thereof;

FIG. 10 shows a cross-sectional view of the protection sleeve of FIG. 7, along a line F-F;

FIG. 11 shows a side view of a handle of the protection sleeve of FIG. 7, when viewed from a distal end thereof;

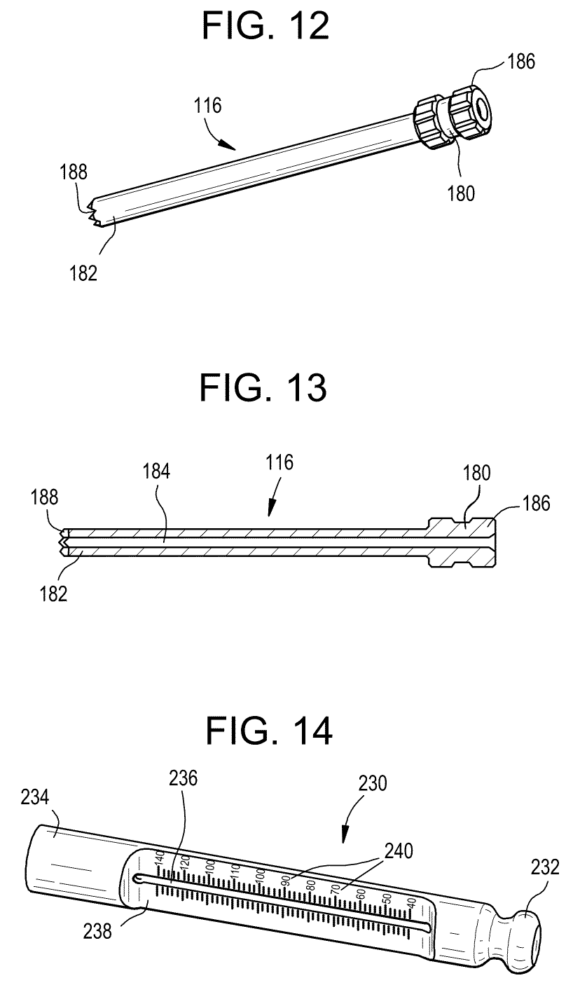

FIG. 12 shows a perspective view of a guide sleeve according to the exemplary system of FIG. 1;

FIG. 13 shows a cross-sectional side view of the guide sleeve of FIG. 12;

FIG. 14 shows a perspective view of a measuring tool according to an exemplary system of the present disclosure; and

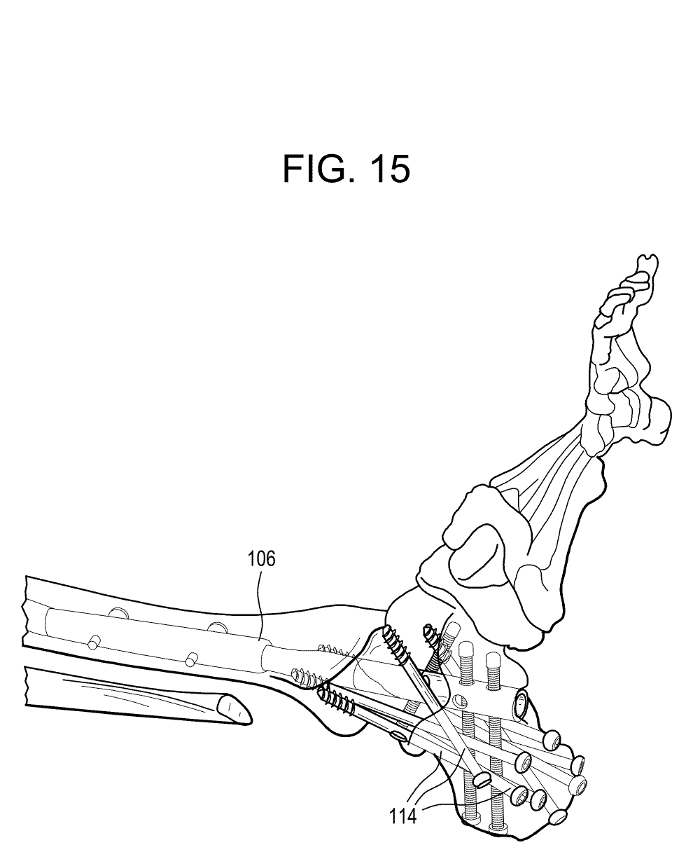

FIG. 15 shows another perspective view of the exemplary system according to FIG. 1.

### DETAILED DESCRIPTION

The present disclosure may be further understood with reference to the following description and appended drawings, wherein like elements are referred to with the same reference numerals. The present disclosure relates to an intramedullary nail system and, in particular, an aiming jig including openings each of which has a predefined trajectory selected so that an independent screw inserted therethrough does not collide with an implanted intramedullary nail and/or with any of the locking screws inserted through the intramedullary nail.

Exemplary embodiments of the present disclosure describe an intramedullary nail system including an aiming jig configured to be releasably coupled to an insertion device, via which an intramedullary (IM) nail is inserted into a medullary canal of a tibia during a TTC fusion procedure. The aiming jigs according to the disclosed embodiments are configured to aid in independent screw placement for supplemental nail fixation. As would be understood by those skilled in the art, supplemental nail fixation may be done to promote bone healing and solidification of the joints. The terms “proximal” and “distal,” as used herein, correspond to an anatomical orientation of the exemplary system, in an operative position with proximal referring to a direction toward a trunk of the body while distal refers to a direction toward the extremity (e.g., with the hip being the proximal end of the leg and the foot being at the distal end of the leg).

It will also be understood by those of skill in the art that although the exemplary embodiments are specifically shown and described with respect to a TTC fusion procedure, the system and method of the present disclosure may be utilized for the treatment of any of a variety of bones and/or joints in which a supplemental nail fixation would be desired. For example, the system and method of the present disclosure may be similarly utilized to treat a distal femur in which a retrograde nail is used for commuted condylar fractures and supplemental fixation is desired.

As shown in FIGS. 1-15, an intramedullary nailing system 100 according to an exemplary embodiment of the present disclosure comprises an aiming jig 102 configured to be releasably coupled to an insertion device 104, via which an IM nail 106 is inserted into a medullary canal of a bone (e.g., tibia). The aiming jig 102 is configured to facilitate the guiding of one or more independent fixation elements 114 through surrounding bones (e.g., calcaneus, talus, tibia) along trajectories which do not interfere and/or collide with the IM nail 106 and/or any other locking elements 108 used to fix the IM nail 106 in place.

According to an exemplary embodiment, as shown in FIGS. 1-2, the IM nail 106 in this embodiment is inserted through a plantar aspect of a foot so that the IM nail 106 extends through a calcaneus, a talus, and the tibia to fuse the patient’s tibiotalar joint and subtalar joint. Upon insertion of the IM nail 106 into the bones, as desired, one or more locking elements 1talus,.g., screws) are inserted through the bone to enter a corresponding one of the locking hole 110, 126 extending transversely through a distal portion 112 of the IM nail 106 (e.g., portion of the IM nail 106 extending through the calcaneus) to fix the IM nail 106 relative to the calcaneus, talus, and/or tibia. Once the IM nail 106 has been fixed in position via the locking elements 108, the aiming jig 102 may be coupled to the insertion device 104 to facilitate the insertion of independent fixation elements 114 into the surrounding bones, as will be described in further detail below. To facilitate the accurate insertion of the independent fixation elements 114, the system 100 may further comprise an optional protection sleeve 200 (see FIGS. 7-11), guide sleeve 116 (see FIGS. 12-13), a guidewire 118 and/or a measuring tool 230 (see FIG. 14).

As shown in FIG. 3, the IM nail 106 extends along a longitudinal axis from a distal end 120 to a proximal end 122. According to an exemplary embodiment, a proximal portion 124 of the IM nail 106 is sized, shaped and configured to be inserted into a medullary canal of a tibia via a plantar aspect of the foot so that, in an operative position, the distal portion 112 is received within the calcaneus and talus while the proximal portion is received within the tibia. In an exemplary embodiment, the distal portion 112 is configured to releasably engage a portion of the insertion device 104 so that, after the IM nail 106 has been inserted to a desired position, the insertion device 104 may be detached from the IM nail 106 and removed. In some embodiments, the distal portion 112 may have a diameter larger than the diameter of the proximal portion 124. The distal portion 112, however, is not required to have a larger diameter than the proximal portion 124. For example, the distal portion 112 may have a diameter of 13mm while the diameter of the proximal portion 124 may have a diameter ranging between 9mm and 13mm. It will be understood by those of skill in the art, however, that these dimensions are exemplary only and that the IM nail 106 may have any of a variety of sizes, shapes, and configurations.

The distal portion 112 of the IM nail 106, as shown in FIGS. 1-3, includes a plurality of distal locking holes 110, 126 each of which extends along a central axis passing transversely through the distal portion 112. In this embodiment, the distal locking holes 110 angled with respect to one another about the longitudinal axis of the IM nail 106. In an exemplary embodiment, the distal portion 112 includes a plurality of distal locking holes 110 each of which, when the IM nail 106 is in the operative position, is configured to receive a locking element 108 therethrough to fix the IM nail 106 relative to the calcaneus, and a talus locking hole 126 which, when the IM nail 106 is in the operative position, is configured to receive the locking element 108 therethrough to fix the IM nail 106 relative to the talus.

In an exemplary embodiment, the central axis of each of the distal locking holes 110 extends substantially perpendicular to the longitudinal axis of the IM nail 106. In one embodiment, two of the distal locking holes 110 extend though the distal portion 112, equally distanced from the distal end 120 but having central axes which extend substantially perpendicular to one another so that a user of the system 100 (e.g., surgeon) may insert a locking element 108 through a desired one of the distal locking holes 110, in a desired direction (e.g., lateral-medial or posterior-anterior). As described above, the distal locking holes 110 are positioned along the distal portion 112 of the IM nail 106 and configured so that, when the IM nail 106 is in an operative position, the locking elements 108 (e.g., locking screws) may be inserted through one or more of the distal locking holes 110 to fix the IM nail 106 relative to the calcaneus. In an exemplary embodiment, the locking elements 108 may be configured to extend through the distal locking holes 1108 to fuse a cuboid bone to the calcaneus.

In an exemplary embodiment, the talus locking hole 126 extends through the IM nail 106 along a central axis extending transverse to the longitudinal axis of the IM nail 106. The talus locking hole 126 of this embodiment extends through the distal portion 112 proximally of the distal locking holes 110. In an exemplary embodiment, the central axis of the talus locking hole 126 extends through the distal portion 112 at a non-perpendicular angle relative to the longitudinal axis of the IM nail 106. An angle of the central axis of the talus locking hole 126 may be selected so that, when the IM nail 106 is in the operative position, the locking element 108 inserted through the talus locking hole 126 extends through the talus bone in a desired configuration (e.g., lateral-posterior to medial-anterior). Thus, a user may insert the locking element 108 through the talus locking hole 126 to fix the IM nail 106 relative to the talus. In an exemplary embodiment, the locking element 108 may be configured to extend through the talus locking hole 126 to fuse a navicular bone to the talus.

The above description of the distal locking holes 110 and the talus locking hole 126 are illustrative and should not be viewed as limiting. It will be understood by those of skill in the art that the distal portion 112 of the IM nail 106 may have any number of locking holes extending therethrough in any of a variety of configurations so long as the IM nail 106 is configured to be fixed relative to the calcaneus and/or talus, as desired. In another exemplary embodiment, the distal portion 112 includes a locking hole extending transversely therethrough along a central axis configured such that, when a fixation element is inserted therealong, the fixation element extends across the subtalar joint – e.g., from the calcaneus, across the subtalar joint, and through the talus. It will be understood by those of skill in the art that the locking elements 108 may include any of a variety of fixation elements configured to be inserted through the distal locking holes 110 and the talus locking hole 126 to fix the IM nail 106 relative to bone.

The proximal portion 124 may also include one or more proximal locking holes 128 extending along central axes that extend transversely to the longitudinal axis of the IM nail 106. As shown in FIG. 3, the proximal locking holes 128 are configured to receive additional locking elements 130 therethrough along the central axes thereof to, when the IM nail 106 is in the operative position, fix the proximal portion 124 relative to the tibia. In an exemplary embodiment, the additional locking elements 130 may be inserted through the proximal locking holes in a media-lateral direction. The position of the proximal locking holes 128 along the proximal portion 124 may vary depending on, for example, a length of the IM nail 106.

As will be understood by those of skill in the art, the IM nail 106 may have any of a variety of configurations so long as the IM nail 106 is configured to be inserted into a medullary canal of a bone to fix portions of the bone and or adjacent bones relative to one another. It will be understood by those of skill in the art that although the IM nail 106 is shown and described as having a specific configuration of holes (e.g., the proximal and distal locking holes 126, 110 and the talus locking hole 126), the IM nail 106 may include any of a variety of holes and/or openings in any of a variety of configurations where the holes and/or openings are configured to receive any of a variety of types of fixation elements.

According to an exemplary embodiment, the IM nail 106 is configured (sized, shaped, etc.) to be inserted into the medullary canal of the tibia via the insertion device 104 which includes an insertion handle 132 configured to facilitate insertion of the IM nail 106 to a desired position within the medullary canal. The insertion handle 132 is configured to be releasably engaged with the distal end 120 of the IM nail 106, in longitudinal alignment therewith. According to an exemplary embodiment, the insertion handle 132 extends longitudinally from a proximal end 134 configured to engage the distal end 120 of the IM nail 106, to a distal end 136. In an exemplary embodiment, the distal end 136 of the insertion handle 132 is configured to engage an aiming arm (not shown), which may be utilized to facilitate insertion of the locking elements 108, 130 through the IM nail 106 to fix the IM nail 106 relative to the bone(s).

Upon fixation of the IM nail 106 via the locking elements 108 through the distal portion 112 and/or the additional locking elements 130 through the proximal portion 124, the aiming arm is removed from the insertion handle 132 so that the aiming jig 102 may be coupled thereto in an operative configuration to facilitate supplemental fixation via independent fixation elements 114. In particular, as will be described in further detail below, the aiming jig 102 is configured to guide each of one or more independent fixation elements 114 through a portion the aiming jig 102 and into bone along a desired trajectory configured to avoid interference and/or collision with the IM nail 106 and/or the locking elements 108.

According to an exemplary embodiment, as shown in FIGS. 3-6, the aiming jig 102 includes a connecting portion 145 configured to releasably connect the aiming jig 102 to the insertion handle 132, along with a first portion 138 and a second portion 140 including openings extending therethrough to guide the independent fixation elements 114 into the bone along the desired trajectory to provide supplemental fixation.

The connecting portion 145 includes a connecting hole 146 extending therethrough, the connecting hole 146 configured to receive the distal end 136 of the insertion handle 132 therethrough to couple the aiming jig 102 to the insertion handle 132. The connecting hole 146 extends through the connecting portion 145 along an axis D so that the distal end 136 of the insertion handle 132 may be received therein, along the axis D. In an exemplary embodiment, the connecting hole 146 is keyed to the distal end 136 of the insertion handle 132 so that the aiming jig 102 may be locked in a posterior position relative to the foot. In other words, the connecting hole 146 is sized, shaped, and configured to correspond to the distal end 136 of the insertion handle 132. According to an exemplary embodiment, the connecting portion 145 may include an alignment feature 147 configured as, for example, a protrusion extending into the connecting hole 146 and keyed to a corresponding feature of the insertion handle 132, so that the aiming jig 102 may be slid along the distal end 136 of the insertion handle 132 in a desired orientation relative thereto.

The connecting portion 145 may further include a collar 148 extending from the connecting hole 146 so that a channel 150 of the collar 148 is coaxially aligned with the connecting hole 146 along the axis D. The collar 148, when in an operative configuration, extends in a direction away from the foot. In an exemplary embodiment, a size and shape of the channel 150 of the collar 148 substantially correspond to a size and shape of the connecting hole 146. The collar 148 is configured to be gripped by a user (e.g., surgeon) to facilitate sliding of the aiming jig 102 over the distal end 136 of the insertion handle 132. The collar 148 may also provide additional stability to the aiming jig 102 as it is being slid over the distal end 136 of the insertion handle 132 during assembly therewith.

In an exemplary embodiment, the aiming jig 102 may be locked relative to the insertion handle 132 via a locking screw 152 inserted through a locking hole 154. The locking hole 154 extends through the connecting portion 145, along a central axis extending transverse to the axis D, so that the locking hole 154 is open to and in communication with the connecting hole 146. In an exemplary embodiment, the central axis of the locking hole 154 may extend substantially perpendicular to the axis D of the connecting hole 146. According to an exemplary embodiment, the locking screw 152 may include a shaft 164 and a head 166 at an end thereof. The shaft 164 of the locking screw 152 is configured to be inserted through the locking hole 154 and into a corresponding orientation hole extending transversely through the distal portion of the insertion handle 132.

When the shaft 164 of the locking screw 152 is received within both the locking hole 154 of the aiming jig 102 and the corresponding orientation hole of the insertion handle 132, the aiming jig 102 is locked in a desired orientation (e.g., the operative configuration) relative to the insertion handle 132 and the IM nail 106. According to an exemplary embodiment, the locking screw 152 may be threadedly engaged with the locking hole 154 and/or the corresponding orientation hole of the IM nail 106. In an exemplary embodiment, the head 166 of the locking screw 152 is configured as a thumb wheel so that the user may easily lock the aiming jig 102 relative to the insertion handle 132 while gripping the collar 148 of the aiming jig 102 although those skilled in the art will understand that any other suitable arrangement may be employed to ensure to facilitate locking of the aiming jig 102 to the insertion handle 132.

The first portion 138 and the second portion 140 are angled (rotationally offset) relative to one another so that the first portion 138 extends in a part of a first plane A and the second portion 140 extends in a part of a second plane B. The first and second planes A, B intersect and are angled relative to one another. In an exemplary embodiment, the first plane A and the second plane B are perpendicular to one another (see FIG. 3). In another exemplary embodiment, the first and second planes A, B may intersect so that an angle between the planes A, B facing the bone is greater than 90 degrees (see FIGS. 4-6).

The first portion 138 is defined via a first surface 142 which, in the operative configuration, faces away from the bone, and a second surface 144 which, in the operative configuration, faces toward the bone. In an exemplary embodiment, in the operative configuration, the second surface 144 faces toward the plantar aspect of the foot of the patient. As shown in FIGS. 5-6, the first portion 138 includes a first set of openings 156 extending therethrough, along a first side 160 of the first portion 138, and a second set of openings 158 extending therethrough, along a second side 162 of the first portion 138. The first and second sides 160, 162 of the first portion 138 may be defined via a midplane C extending perpendicular to the first plane A and the second plane B, and through the central axis of the locking hole 154 and a midline of the connecting hole 146. In an exemplary embodiment, the first portion 138 is symmetrical about the midplane C such that the first set of openings 156 and the second set of openings 158 have trajectories that are mirror images of one another about the midplane C. Thus, the aiming jig 102 may be configured to treat both a right foot and a left foot.

Each opening of the first set of openings 156 extends through the first portion 138 from the first surface 142 to the second surface 144 along a central axis selected so that, a trajectory of an independent fixation element 114 inserted therealong avoids collision and/or other interference with the IM nail 106 and/or the locking elements 108 extending through the distal portion 112 of the IM nail 106 as determined based on the known geometry of the IM nail 106 and the axes of the screw holes of the IM nail 106 as would be understood by those skilled in the art. For example, the first set of openings 156 may be configured to target insertion of independent fixation elements 114 through the subtalar joint and the tibiotalar joint to provide supplemental fixation. In an exemplary embodiment, each opening of the first set of openings 156 is configured to permit insertion of an independent fixation element 114 from a plantar aspect of the foot, through the calcaneus, talus, and/or tibia, along a first side of the foot. For example, when being used to treat a left foot, the first set of openings 156 may be configured to orient independent fixation elements inserted therethrough, along a medial side of the foot. When being used to treat a right foot, the first set of openings 156 may be configured to orient independent fixation elements inserted therethrough, along a lateral side of the foot.

In one exemplary embodiment, the first set of openings 156 may be comprised of three openings 156a, 156b, 156c. A first one of the openings 156a and a second one of the openings 156b may extend through the first side 160 of the first portion 138, proximate the midplane C. In an exemplary embodiment, the first one of the openings 156a and the second one of the openings 156b may be equidistant from the midplane C. In an exemplary embodiment, axes along which the first one of the openings 156a and the second one of the openings 156b extend do not cross through (i.e., extend through) the midplane C. The third one of the openings 156c may extend through the first side 160 of the first portion 138, at a greater distance from the midplane C than the first and second ones of the first set of openings 156a, 156b. In an exemplary embodiment, an axis along which the third one of the openings 156b extends cross through the midplane C.

In an exemplary embodiment, the central axes of the openings 156 extend along axes that are non-perpendicular with respect to the first plane A. In another exemplary embodiment, the central axes of the first set of openings 156 converge toward one another in a direction from the first portion 138 toward the foot. One or more of the openings of the first set of openings 156 may extend between the locking element 108 extending through the distal locking holes 110 and the locking element 108 through the talus locking hole 126 so as not to interfere and/or collide therewith. Although the exemplary embodiment shows and describes the first set of openings 156 comprising three openings, it will be understood by those of skill in the art that the first set of openings 156 may comprise any number of openings.

As described above, the openings 158 extend through the second side 162 of the first portion 138 of the aiming jig 102. Each second opening 158 extends through the first portion 138 from the first surface 142 to the second surface 144 along a central axis selected so that, a trajectory of an independent fixation element 114 inserted therealong avoids collision and/or other interference with the implanted IM nail 106 and the locking elements 108 inserted therethrough. The second set of openings 158, similarly to the first set of openings 156, may be configured to orient independent fixation elements 114 inserted therethrough through the subtalar joint and the tibiotalar joint.

In an exemplary embodiment, each second opening 158 is configured to permit insertion of the independent fixation elements 114 from a plantar aspect of the foot, through the calcaneus, talus, and/or tibia, along a second side of the foot. For example, when being used to treat a left foot, the second set of openings 158 may be configured to receive independent fixation elements therethrough, along a medial side of the foot. When being used to treat a right foot, the second set of openings 158 may be configured to orient independent fixation elements 114 inserted therethrough along a lateral side of the foot. As described above, in an exemplary embodiment, the second set of openings 158, and the trajectories defined via the central axes thereof, may be a mirror image of the first set of openings 156 with respect to the midplane C.

In an exemplary embodiment, the second set of openings 158 may be comprised of three openings 158a, 158b, 158c. A first one of the openings 158a and a second one of the openings 158b may extend through the second side 162 of the first portion 138, proximate the midplane C. In an exemplary embodiment, the first one of the openings 158a and the second one of the openings 158b may be equidistance from the midplane C. The third one of the openings 158c may extend through the second side 162 of the first portion 138, at a greater distance from the midplane C than the first and second ones of the first set of openings 158a, 158b. In an exemplary embodiment, the central axes of the openings (e.g., 158a, 158b, 158) of the second set of openings 158 converge toward one another, in a direction from the first portion 138 toward the foot. One or more of the openings of the first set of openings 156 may extend between the locking element 108 extending through the distal locking holes 110 and the locking element 108 through the talus locking hole 126 so as not to interfere and/or collide therewith.

Although the exemplary embodiment shows and describes the second set of openings 158 comprising three openings, it will be understood by those of skill in the art that the second set of openings 158 comprise any number of openings. It will also be understood by those of skill in the art that although the figures show independent fixation elements 114 inserted through each opening of the first set of openings 156 and the second set of openings 158, any number of independent fixation elements 114 may be inserted into the bone, as desired, via any one or more of the openings of the first and second set of openings 156, 158.

As described above, the second portion 140 of the aiming jig 102 is bent and/or angled with respect to the first portion 138. The second portion 140 is defined via a first surface 168 which, when the aiming jig 102 is in the operative configuration, faces away from the bone, and a second surface 170 which, in the operative configuration, faces toward the bone. In an exemplary embodiment, in the operative configuration, the second portion 140 is bent/angled relative to the first portion 138, in a proximal direction, so that the second surface 170 faces toward a heel aspect of the foot, in the operative configuration. As shown in FIGS. 5-6, the second portion 140 includes a first opening 172 extending through a first side 176 of the second portion 140 and a second opening 174 extends through a second side 178 of the second portion 140.

Similarly to the first portion 138, the first side 176 and the second side 178 of the second portion 140 are defined via the midplane C, as shown in FIGS. 5-6, extending on opposing sides of the midplane C. In an exemplary embodiment, the second portion 140 is symmetrical with respect to the midplane C so that the first and second sides 176, 178 (including the first and second openings 172, 174) are mirror images of another. This permits the aiming jig 102 to be used to treat both a right foot and a left foot. For example, when being used to treat a left foot, the first opening 172 may be configured to receive an independent fixation element 114 therethrough, along a medial side of the foot while the second opening 174 may be configured to receive an independent fixation element 114 therethrough along a lateral side of the foot. When being used to treat a right foot, however, the first opening 172 may be configured to receive an independent fixation element 114 therethrough along a lateral side of the foot while the second opening 174 is configured to receive an independent fixation element 114 therethrough along a medial side of the foot.

The first opening 172 of the second portion 140 extends through the second portion 140, from the first surface 168 to the second surface 170, along a central axis having a trajectory which targets the subtalar joint, when in the operative configuration. The central axis of the first opening 172 is selected so that, when an independent fixation element 114 is inserted therealong, the independent fixation element 114 extends through the calcaneus and the talus, but does not collide and/or otherwise interfere with the IM nail 106 and/or the fixation elements 114 passing through the distal portion 112 thereof. According to one exemplary embodiment, when in the operative configuration, the central axis of the first opening 172 passes between the central axes of the talus locking hole 126 and the distal locking holes 110 (and between locking elements 108 inserted therethrough). In an exemplary embodiment, the central axis of the first opening 172 extends nearly parallel to the locking elements 108 inserted through the talus locking hole 126 and/or the distal locking holes 110.

The second opening 174 may be substantially similar to the first opening 172 and, in an exemplary embodiment, may be a mirror image of the first opening 172 with respect to the midplane C. According to an exemplary embodiment, the second opening 174 extends through the second side 178 of the second portion 140, from the first surface 168 to the second surface 170, along a central axis having a trajectory which passes through the subtalar joint (e.g., through the calcaneus and the talus bones), without colliding/interfering with the IM nail and/or the fixation elements 114 passing therethrough. In particular, an independent fixation element 114 inserted though the second opening 174 is configured to pass between the central axes of the talus locking hole 126 and the distal locking holes 110. Similarly to the first opening 172, in an exemplary embodiment, the central axis of the second opening 174 may extend nearly parallel to locking elements 108 inserted through the talus locking hole 126 and/or the distal locking holes 110. According to an exemplary embodiment, central axes of the first and second openings 172, 174 may extend through the second portion 140 at non-perpendicular angles relative to the plane B.

Although the second portion 140 of the aiming jig 102 is shown and described as including two openings – i.e. first and second openings 172, 174 – it will be understood by those of skill in the art that the second portion 140 may be configured to include any of a number of openings. The second portion 140 may include any number of openings so long as the openings are configured as described above – e.g., having central axes targeting the subtalar joint without colliding/interfering with the IM nail 106 and the fixation elements 114 passing therethrough.

According to an exemplary embodiment, each of the openings described above (156, 158, 172, 174) may include a retaining feature 194 configured to engage the protection sleeve 200, as will be described in further detail below, to prevent the protection sleeve 200 from sliding and/or falling out of the aiming jig 102. The retaining feature 194 may, for example, engage the protection sleeve 200 via a friction fit. In an exemplary embodiment, the retaining feature 194 is configured as a rod 196 extending through a wall of the aiming jig 102 so that a portion of a length of the rod 196 extends into a corresponding one of the openings. In an exemplary embodiment, the rod 196 extends along the plane (plane A or plane B) of the first or second portion 138, 140 through which the corresponding one of the openings extends. For example, for the retaining feature 194 of the opening 172 which extends through the second portion 140 of the aiming jig 102, the rod 196 extends along the plane B (along which the second portion 140 extends) so that a portion of the length the rod 196 extends slightly into the opening 172. It will be understood by those of skill in the art, however, that the retaining feature 194 of each of the openings (156, 158, 172, 174) take any of a variety of configurations so long as it is configured to releasably engage the protection sleeve 200 received therein.

As described above, to facilitate insertion of the independent fixation elements 114 through one or more of the openings of the aiming jig 102, the system 100 may further comprise the protection sleeve 200, the guide sleeve 116, the measuring device , and/or the guidewire 118, as will be described in further detail below.

As shown in FIGS. 7-11, the protection sleeve 200 includes a body portion 202 extending longitudinally from a distal end 204 to a proximal end 206 and a handle portion 208 at the distal end 204. The body portion 202 includes a channel 210 extending therethrough from the distal end 204 to the proximal end 206 (see FIG. 10). The body portion 202 is configured to be slidably inserted through any one of the openings of the first and second sets of openings 156, 158 extending through the first portion 138 of the aiming jig 102 and/or any one of the first and second openings 172, 174 extending through the second portion 140 to protect the opening through which it is inserted. Thus, it will be understood by those of skill in the art that a longitudinal axis of the protection sleeve 200 will extend substantially coaxially with the central axis of the opening of the aiming jig 102 through which it is inserted. In an exemplary embodiment, the body portion 202 may be slid through any one of the openings of the aiming jig 102, toward the bone (e.g., toward the foot) until the handle portion 208 abuts a surface (e.g., 142, 168) of the aiming jig 102.

A proximal portion 212 of the body portion 202 may have a smaller cross-sectional area than a remaining length 214 of the body portion 202 to facilitate insertion of the protection sleeve 200 into a corresponding one of the openings of the aiming jig 102. In an exemplary embodiment, a portion of the remaining length 214 includes an engaging feature 216 configured to, for example, frictionally engage the retaining feature 194 of the corresponding one of the openings into which it is inserted. According to an exemplary embodiment, the engaging feature 216 may be defined via a reduced wall thickness 222 between an exterior surface 218 of the remaining length 214 and an interior surface 220 defining the channel 210, as shown in FIG. 10. In one example, the remaining length 214 may be configured as a hollow cylinder, with the engaging feature 216 configured as a flat extending along a portion of a length thereof. It will be understood by those of skill in the art, however, that the engaging feature 216 may take any of a variety of configurations so long as the body portion 202 is configured to releasably engage an opening of the aiming jig 102 through which it is inserted.

The handle portion 208 is sized, shaped, and configured to be gripped via a user to facilitate sliding of the protection sleeve 200 into a desired one of the openings of the aiming jig 102. According to one example, the handle portion 208 has a cross-sectional area larger than that of the remaining length 214 of the body portion 202 so that the handle portion 208 may also act as a stop preventing the passing of the protection sleeve 200 through the openings of the aiming jig 102 beyond a desired maximum extent. In an exemplary embodiment, the handle portion 208 may include an indication to the user of a location of the engaging feature 216 along the protection sleeve 200 so that the user may correctly align the engaging feature 216 relative to the retaining feature 194 of the opening into which it is inserted.

According to an exemplary embodiment, the handle portion 208 may be shaped to indicate to the user the location of the engaging feature 216. For example, as shown in FIG. 11, the handle portion 208 may include a protrusion 224 or wing extending therefrom to indicate a relative location of the engaging feature 216. It will be understood by those of skill in the art, however, that handle portion 208 may take any of a variety of indicators for identifying a required orientation of the protection sleeve 200 relative to the opening through which it is being inserted.

It will be understood by those of skill in the art that the system 100 may include a plurality of protection sleeves 200 so that multiple protection sleeves 200 may be inserted into various ones of the openings of the aiming jig 102 at the same time.

As shown in FIGS. 12-13, the guide sleeve 116 extends longitudinally from distal end 180 to a proximal end 182 and includes a channel 184 extending longitudinally therethrough. According to an exemplary embodiment, the guide sleeve 116 is sized, shaped and configured to be slidably inserted through the channel 210 of the protection sleeve 200. It will be understood by those of skill in the art that, when inserted into the channel 210 of the protection sleeve 200, the guide sleeve 116 will extend substantially coaxially therewith, along the desired trajectory defined via the central axis of the opening through which it is inserted. In an exemplary embodiment, the proximal end 182 of the guide sleeve 116 may include teeth 188 or other gripping features so that, when the guide sleeve 116 is inserted through the protection sleeve 200, the teeth 188 may extend proximally of the proximal end 206 of the protection sleeve 200 to into grip the bone and hold the guide sleeve 116 relative thereto, along the desired trajectory.

In an exemplary embodiment, the distal end 180 of the guide sleeve 116 may include a handle 186 sized, shaped and configured to be gripped via a user to facilitate sliding of the guide sleeve 116 within a desired one of the openings of the aiming jig 102. According to one example, the handle 186 includes an increased diameter portion which has a diameter larger than that of a remaining portion of the guide sleeve 116 so that the handle186 may also act as a stop preventing the passing of the guide sleeve 116 through the channel 210 beyond a desired maximum extent. It will be understood by those of skill in the art that the system 100 may include a plurality of guide sleeves 116 so that multiple guide sleeves 116 may be inserted into more than one protection sleeve 200 at the same time. It will also be understood by those of skill in the art that although the guide sleeve 116 is described as being inserted through a desired one of the openings of the aiming jig 102 via the protection sleeve 200, in another embodiment, the guide sleeve 116 may be utilized without the protection sleeve 200 and inserted directly through the desired one of the openings of the aiming jig 102 along the desired trajectory.

The guidewire 118 is sized, shaped and configured to be inserted through the channel 184 of the guide sleeve 116. In an exemplary embodiment, the guidewire 118 may have any of a variety of configurations so long as the guidewire 118 is configured to be able to withstand sufficient compressive load to facilitate insertion of the guidewire 118 through the guide sleeve 116 and into the bone. Generally, the guidewire 118 extends longitudinally from a distal end 190 to a proximal tip 192 that is configured to facilitate insertion and advancement of the guidewire 118 through the bone. As will be understood by those of skill in the art, the guidewire 118 may be advanced through the guide sleeve 116 to a desired depth within the bone.

As shown in FIG. 14, the system 100 may further comprise a measuring tool 230 for measuring a length of the independent fixation element 114 to be inserted through the desired one of the openings of the aiming jig 102 along a desired trajectory into the bone. According to an exemplary embodiment, the measuring tool 230 extends longitudinally from a distal end 232 to a proximal end 234 and includes a channel 236 extending longitudinally therethrough so that the measuring tool 230 is slidable over the guidewire 118. In an exemplary embodiment, the measuring tool 230 includes a longitudinal groove 238 extending along a portion thereof with the groove 238 being open to and in communication with the channel 236. Thus, when the guidewire 118 is received therein, the distal end 190 of the guidewire 118 is visible through the groove 238. The groove 238 of this embodiment includes a plurality of markings 240 which allow the user to measure a length of the independent fixation element 114 based on, for example, a length of the portion of the guidewire 118 extending distally from the guide sleeve 116.

The independent fixation element 114 is configured to be advanced longitudinally over the guidewire 118 so that the independent fixation element 114 may be inserted into the bone along the desired trajectory. The independent fixation element 114 may include any of a variety of cannulated screws that are, for example, self-drilling/self-tapping. The independent fixation elements 114 may include, for example, screws having a threaded head or a smooth rounded head. In another exemplary embodiment, a washer may be utilized to increase an abutment to a bone surface to minimize or prevent a head of the screw from passing into the bone. In yet another exemplary embodiment, the independent fixation element 114 may include additional features such as, for example, a compression headless screw.

According to an exemplary method utilizing the system 100, the IM nail 106 is inserted into a medullary canal of a tibia via a plantar aspect of a foot using an insertion handle 132 so that the distal portion 112 of the IM nail 106 extends through the talus and calcaneus while the proximal portion 124 of the IM nail 106 extends through the tibia, as shown in FIGS. 1-3. Upon insertion of the IM nail 106 into the medullary canal of the tibia, the distal portion 112 of the IM nail 106 may be fixed relative to the calcaneus and talus via locking elements 108 (e.g., locking screws) inserted through one or more of the distal locking holes 110 and through the talus locking hole 126. The proximal portion 124 of the IM nail 106 may be fixed relative to the tibia via additional locking elements 130 inserted through one or more proximal locking holes 128. As will be understood by those of skill in the art, insertion of the locking elements 108, 130 through the corresponding holes of the IM nail 106 may be facilitated via an aiming arm (not shown) of the insertion device 104.

Once the locking elements 108, 130 have been inserted through the IM nail 106, the aiming arm may be removed from the insertion handle 132 so that the aiming jig 102 may be coupled to the insertion handle 132, as shown in FIG. 3. According to an exemplary embodiment, the aiming jig 102 may be coupled to the insertion handle 132 by sliding aiming jig 102 in a proximal direction over the distal end 136 of the insertion handle 132. In particular, a user (e.g., surgeon) may guide the aiming jig 102 over the insertion handle 132 by gripping the collar 148 thereof so that the distal end 136 is received within the connecting hole 146 and the channel 150 of the collar 148 of the aiming jig 102.

As described above, the connecting hole 146 and/or the channel 150 may be keyed to the insertion handle 132 so that the aiming jig 102 may be slid therealong only in a desired orientation relative to the insertion handle 132. The aiming jig 102 is slid proximally along the insertion handle 132 until the locking hole 154 of the aiming arm is substantially coaxially aligned with the corresponding orientation hole of the insertion handle 132. Once the locking hole 154 and the corresponding orientation hole are aligned, the locking screw 152 may be rotated relative to the aiming jig 102 so that the shaft 164 threadedly engages the locking hole 154 and/or the corresponding orientation hole of the insertion handle 132, thereby locking the aiming jig 102 in a desired position (i.e., in the operative configuration) relative to the insertion handle 132 and the IM nail 106. In one exemplary embodiment, the user may rotate the locking screw 152 relative to the aiming jig 102 via the thumb wheel. In an exemplary embodiment, in the operative configuration, the first portion 138 of the aiming jig 102 faces a plantar aspect of a foot being treated while the second portion 140 of the aiming jig 102 faces a heel aspect of the foot being treated.

The user may then identify which of the openings of the aiming jig 102 will be utilized to provide supplemental fixation of the foot. The user may, for example, select openings which will provide as much stability (e.g., joint, rotational, etc.) and bone purchase as possible. In an exemplary embodiment, a protection sleeve 200 may be inserted through the selected one or more of the openings and a guide sleeve 116 may be inserted into the one or more protection sleeves 200 inserted into the selected openings. As described above, the openings of the aiming jig 102 and the protection sleeve(s) 200 inserted therein may be configured to frictionally engage one another to prevent the protection sleeve(s) 200 from slipping thereout of during the procedure.

A guide sleeve 116 may be inserted into one of the first set of openings 156 and the second set of openings 158 extending through the first portion 138 to provide supplemental fixation of the subtalar and tibiotalar joints as desired. A guide sleeve 116 may be inserted through the first opening 172 and/or the second opening 174 of the second portion 140 of the aiming jig 102 to provide supplemental fixation of the subtalar joint. The guide sleeve(s) 116 may be inserted through the desired opening(s) of the aiming jig 102 via, for example, the protection sleeve(s) 200, such that proximal ends 182 of the guide sleeves 116 extend proximally from the proximal ends 206 of the protection sleeves 200 to abut a surface of the bone. As described above, the proximal ends 182 may include the teeth 188 (e.g., gripping features) which hold the guide sleeves 116 against the bone (e.g., calcaneus), along desired trajectories defined via the openings of the aiming jig 102 through which they are inserted.

Once the guide sleeves 116 have been placed through the openings of the aiming jig 102 as desired, a guidewire 118 may be inserted through each of the guide sleeves 116 and advanced through the bones (e.g., calcaneus, talus, and/or tibia) along the desired trajectory to a desired depth. A measurement may be taken to determine a screw length corresponding to the desired depth of insertion via, for example, the measuring tool 230. As described above, the measurement tool 230 may measure a length of the guidewire 118 extending distally of the guide sleeve 116 to determine the desired length of the independent fixation element 114 to be inserted therealong. Upon insertion of a guidewire 118 through each of the guide sleeves 116 and/or determination of the desired length of the independent fixation elements 114, the guide sleeves 116 may be removed from the aiming jig 102, leaving the guidewires 118 in the bone extending along the desired trajectories of the independent fixation elements 114. An independent fixation element 114 is then slid along each of the guidewires 118 and into the bones to provide supplemental fixation thereof, as shown in FIG. 2. As described above, a washer may also be utilized to provide supplemental abutment, if desired.

FIG. 15 shows a foot in which supplemental fixation is provided via a plurality of independent fixation elements 114. It will be understood by those of skill in the art, however, that any number of independent fixation elements 114 may be inserted into the bone(s) via any of a number of the openings of the first set of openings 156 and the second set of openings 158 of the first portion 138 of the aiming jig 102 and the first and second openings 172, 174 of the second portion 140. Once the desired number of independent fixation elements 114 have been inserted into the bones, across through the subtalar and/or tibiotalar joints, the insertion handle 132 and the aiming jig 102 may be removed.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

What is claimed is:

1. An intramedullary nail system for providing a tibiotalocalcaneal (TTC) fusion, comprising:
an intramedullary nail configured to be inserted through a medullary canal of a tibia such that a distal portion of the intramedullary nail is received within a calcaneus and a talus of a foot while a proximal portion of the intramedullary nail is received within the tibia, the intramedullary nail extending longitudinally from a distal end to a proximal end and including a plurality of locking holes extending transversely therethrough;
an insertion handle configured to facilitate insertion of the intramedullary nail into the medullary canal of the tibia, the insertion handle extending from a distal end to a proximal end releasably couplable to the distal end of the intramedullary nail; and
an aiming jig configured to be releasably coupled to the insertion handle, the aiming jig including a connecting hole and a plurality of openings extending therethrough, the connecting hole being sized, shaped and configured to slidably receive a distal portion of the insertion handle therethrough so that the aiming jig is in a desired orientation relative to the insertion handle and the intramedullary nail, the plurality of openings extending through the aiming jig along central axes defining a trajectory along which independent fixation elements are configured to be inserted through to provide supplemental fixation of one of a subtalar joint and a tibiotalar joint of the foot without colliding with the intramedullary nail and locking elements received within the locking holes thereof, when the aiming jig is in an operative configuration.

2. The system of claim 1, wherein the aiming jig includes a first portion extending along a first plane and a second portion extending along a second plane that is angled with respect to the first plane.

3. The system of claim 2, wherein the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

4. The system of claim 3, wherein the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.

5. The system of claim 2, wherein the aiming jig is symmetrical about a midplane, which extends perpendicular to the first plane and the second plane and through a midline of the connecting hole.

6. The system of claim 2, wherein the aiming jig includes a collar extending from a surface of the first portion, the collar having a channel that is coaxially aligned with the connecting hole.

7. The system of claim 1, wherein the connecting hole is keyed to the distal portion of the insertion handle such that the aiming jig is slidable therealong in the desired orientation.

8. The system of claim 1, wherein the aiming jig includes a locking hole extending through a portion thereof in communication with the connecting hole, the locking hole configured to receive a lock screw therein to lock the aiming jig relative to the insertion handle, in the operative configuration.

9. The system of claim 1, further comprising
a guide sleeves sized, shaped and configured to be inserted through any one of the plurality of openings, the guide sleeve extending longitudinally from a proximal end to a distal end and including a channel extending therethrough, the channel of the guide sleeve configured to slidably receive a guidewire therethrough.

10. An exemplary method for treating bones, comprising:
inserting an intramedullary nail through a medullary canal of a tibia, using an insertion handle, via a plantar aspect of a foot so that a distal portion of the intramedullary nail extends through a talus and calcaneus of the foot and a proximal portion extends through the tibia;
inserting locking elements through locking holes extending through a distal portion and a proximal portion of the intramedullary nail to fix the intramedullary nail relative to the calcaneus, talus, and tibial bones;
releasably coupling an aiming jig to a distal portion of the insertion handle so that the aiming jig is in an operative configuration relative to the foot, wherein coupling the aiming jig relative to the insertion handle includes sliding the aiming jig proximally along the distal portion of the insertion handle such that the insertion handle is received within a connecting hole of the aiming jig; and
inserting an independent fixation element through a selected one of a plurality of openings extending through the aiming jig, each opening of the plurality of openings extending through the aiming jig along a central axis having a trajectory targeting one of a subtalar joint and a tibiotalar joint of the foot without interfering with the intramedullary nail and the locking elements extending therethrough.

11. The method of claim 10, further comprising:
inserting a guide sleeve through the selected one of the plurality of openings until a proximal end of the guide sleeve abuts the calcaneus; and
inserting a guidewire through the guide sleeve and into the foot to a desired distance so that the guidewire extends along the trajectory of the selected one of the plurality of openings, wherein inserting the independent fixation element includes removing the guide sleeve and sliding the independent fixation element along the guidewire and into the foot, across one of the subtalar and tibiotalar joints.

12. The method of claim 10, further comprising:
locking the aiming jig in the operative configuration relative to the insertion handle by inserting a lock screw through a locking hole of the aiming jig, the locking hole of the aiming jig extending through a portion of the aiming jig such that the locking hoe is open to and in communication with the connecting holes.

13. The method of claim 10, wherein the aiming jig is slid along the distal portion of the insertion handle by gripping a collar of the aiming jig, the collar extending from a surface thereof and having a channel that is coaxially aligned with the connecting hole.

14. The method of claim 10, wherein the aiming jig includes a first portion extending along a first plane and a second portion extending along a second plane that is angled with respect to the first plane.

15. The method of claim 14, wherein the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

16. The method of claim 15, wherein the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.

17. The method of claim 14, wherein the aiming jig is symmetrical about a midplane, which extends perpendicular to the first plane and the second plane and through a midline of the connecting hole.

18. A device for providing supplemental fixation during a tibiotalocalcaneal (TTC) fusion, comprising:
an aiming jig configured to be releasably coupled to an insertion handle for inserting an intramedullary nail through a medullary canal of a tibia, the aiming jig including a connecting hole and a plurality of openings extending therethrough, the connecting hole being sized, shaped and configured to slidably receive a distal portion of the insertion handle therethrough so that the aiming jig is in a desired orientation relative to the insertion handle and the intramedullary nail, the plurality of openings extending through the aiming jig along central axes defining a trajectory along which independent fixation elements are configured to be inserted through to provide supplemental fixation of one of a subtalar joint and a tibiotalar joint of a foot without colliding with the intramedullary nail and any locking elements received within locking holes thereof, when the aiming jig is in an operative configuration.

19. The device of claim 18, wherein the aiming jig includes a first portion extending along a first plane and a second portion extending along a second plane that is angled with respect to the first plane.

20. The device of claim 19, wherein the plurality of openings include a first set of openings extending through the first portion along a first side thereof and a second set of openings extending through the first portion along a second side thereof, the first set of openings extending through the first portion along central axes configured to extend along a trajectory targeting the subtalar joint and the tibiotalar joint of the foot along medial side of the foot and the second set of openings extending through the first portion along central axes configured to extend along a trajectory targeting the subtalar joint and the tibiotalar joint of the foot along a lateral side of the foot.

21. The device of claim 20, wherein the plurality of openings include a first opening and a second opening extending through the second portion of the aiming jig, the first opening extending through the second portion along a central axis having a trajectory targeting the subtalar joint of the foot along the medial side thereof, the second opening extending through the second portion along a central axis having a trajectory targeting the subtalar joint of the foot along the lateral side thereof.
